(19) United States
(12) Patent Application Publication
(10) Pub. No.: US 2016/0007882 A1
(43) Pub. Date: Jan. 14, 2016

(54) DEVICE AND METHOD FOR LUNG MEASUREMENT

(71) Applicant: UNIVERSITY OF VERMONT AND STATE AGRICULTURAL COLLEGE, Burlington, VT (US)

(72) Inventor: Jason H.T. Bates, South Burlington, VT (US)

(21) Appl. No.: 14/764,077

(22) PCT Filed: Jan. 29, 2014
(86) PCT No.: PCT/US14/13695

§ 371 Date: Jul. 28, 2015

Related U.S. Application Data
(60) Provisional application No. 61/757,987, filed on Jan. 29, 2013.

(51) Int. Cl. A61B 5/087; A61M 16/04; A61M 16/08

(52) U.S. Cl. CPC A61B 5/087; A61M 16/08; A61M 16/04; A61M 2016/0027

(57) ABSTRACT

A device and method for measuring the breathing of an individual being mechanically ventilated is presented, the device having a conduit configured as a Venturi tube with a gas flow path therethrough. Pressure transducers are located at positions in the gas flow path such that flow rate and/or other flow parameters may be determined.

### CROSS-REFERENCE TO RELATED APPLICATIONS

The present application claims the benefit of the earlier filing date of U.S. Provisional Patent Application No. 61/757,987, filed Jan. 29, 2013, now pending, the disclosure of which is incorporated herein by this reference.

### STATEMENT REGARDING FEDERALLY SPONSORED RESEARCH

This disclosure was made with government support under contract no. GM-103532 awarded by the National Institutes of Health. The government has certain rights in the disclosure.

### FIELD OF THE INVENTION

The disclosure relates to devices and methods for use with medical ventilators.

### BACKGROUND OF THE INVENTION

Many hospitals in the United States and around the world maintain intensive care units (ICU's) in which very sick patients are provided individual attention around the clock. Many of these patients cannot breath on their own and so are connected to mechanical ventilators that provide periodic episodes of positive pressure in order to inflate the lungs via an endotracheal tube—a plastic tube approximately 20 cm in length with an internal diameter of roughly a centimeter that is lodged in the trachea via the mouth and sealed in place with an inflatable plastic cuff that prevents air leaks.

In many ICU patients, the mechanical properties of the lungs become altered as a result of their disease processes. Essentially what this means is that the lungs either become stiffer than usual and thus more difficult to inflate, or the airways of the lungs become narrower than usual and thus more resistive to gas flow. These changes in mechanical properties affect the mechanical properties of the entire respiratory system (lungs plus chest) and can be quantified, using well-established signal processing methods, from measurements of pressure and flow entering the lungs provided that the patient is not trying to breathe on their own.

If spontaneous breathing efforts are being made by the patient, then the flow measured at the entrance to the tracheal opening is the result of two pressure sources, namely the external source and the patient's own respiratory muscle. The pressure generated by the latter is unknown, so it becomes impossible to estimate the parameters of respiratory mechanics accurately unless additional steps are taken. There are two approaches for solving this problem. The first approach is to measure the pressure in the pleural space between the lungs and the chest wall, as this allows the pressure across the lungs alone to be determined form the difference between pleural pressure and the pressure at the entrance to the endotracheal tube. A surrogate for pleural pressure is the pressure in the esophagus, which can be obtained with an esophageal balloon catheter system. Esophageal catheters are not dangerous, but are cumbersome and poorly tolerated by patients and their families, so it unlikely to succeed as a routine investigative method.

The second approach, when a patient is making breathing efforts, is to apply external oscillations in flow at frequencies that are above the frequency of mechanical ventilation. These additional frequencies may be at or above about 5 Hz. In this way, oscillations in the measured pressure signal at or above 5 Hz are known to be caused by the externally applied flow oscillations at the corresponding frequency and are not the result of actions of the respiratory muscles.

While the use of high frequency oscillations does not allow the distinction of the mechanical properties of the lungs from those of the surrounding chest wall, as is the case with the use of esophageal pressure, the vast majority of important clinical changes in mechanical function (for patients in an ICU) take place in the lungs themselves. Therefore, a measurement of a change in respiratory mechanics is useful as to show changes in lung mechanics.

Also, measurements using the second approach can be made in a noninvasive fashion by adding small-amplitude high-frequency oscillations on top of the normal flow and pressure waveforms applied to a patient's lungs by a conventional mechanical ventilator.

This second approach is generally referred to as the forced-oscillation technique. This technique has been applied previously in clinical situations, including mechanically ventilated patients. However, the previous devices used to achieve this have been experimental prototypes that are cumbersome to set up and which do not ensure that the subject's lungs are isolated from possible infection from the oscillatory device. As such, previous devices have not been accepted as routine clinical tools.

### BRIEF SUMMARY OF THE INVENTION

The present disclosure uses a forced-oscillation technique—applying additional high-frequency oscillations to pressure and flow entering the lungs.

The present disclosure may be embodied as a device for measuring the breathing of an individual being mechanically ventilated, the device having a conduit configured as a Venturi tube with a gas flow path therethrough. A first pressure transducer is positioned in the device at a location along the gas flow path with a first inner diameter (e.g., an inlet diameter) to measure the pressure within the conduit. A second pressure transducer is positioned in the device at a second location where the conduit has a restriction diameter to measure the pressure within the conduit at the second location. A third pressure transducer is positioned in the device at a third location where the conduit has an outlet diameter. The third pressure transducer is configured to measure the pressure of the breathing gas at the third location.

The device also has a measurement orifice between an inlet of the conduit and the first location. The measurement orifice is configured to impart an oscillating pressure on the breathing gas by way of an oscillation source. The device may comprise a diaphragm for isolating the breathing gas of the gas flow path from ambient or other gases which may be present at the measurement orifice. The device may further comprise a housing in pneumatic communication with the measurement orifice. A controller may communicate with the pressure transducers to determine the flow of breathing gas through the conduit.

The present disclosure may be embodied as a method for measuring the lung mechanical function of a patient breathing with the assistance of a ventilator comprising the step of providing a flow meter in the gas flow path. An oscillation in the pressure of the patient's breathing gas is caused and the flow of breathing gas is measured. The portion of breathing gas attributable to the patient's breathing is determined using the measured flow.

### DESCRIPTION OF THE DRAWINGS

For a fuller understanding of the nature and objects of the disclosure, reference should be made to the following detailed description taken in conjunction with the accompanying drawings, in which:

FIG. 1 is a cross-section view, along a longitudinal axis of the device according to an embodiment of the present disclosure;

FIG. 2 is a cross-section view of the device of FIG. 1 taken along a plane perpendicular to the longitudinal axis;

FIG. 3 a diagram of the a sleeve-shaped diaphragm according to an embodiment of the present disclosure;

FIG. 4 is an elevation view of a device according to another embodiment of the present disclosure;

FIG. 5 depicts one configuration of a device according to the present disclosure in relation to a ventilator circuit of a patient;

FIG. 6 is a diagram of a device according to another embodiment of the present disclosure;

FIG. 7 is a view of a model used to simulate a device of the present disclosure;

FIG. 8 depicts comparisons of pressure differentials and flow rates for an input oscillatory flow with a frequency of 5 Hz (forward flow was defined as that from inlet to outlet, and pressures differentials shown are negatives of dp₂₁ and dp₂₃, i.e., p₁-p₂ and p₃-p₂);

FIG. 9 are graphs comparing predicted and actual flow rates for oscillatory flow frequencies of 1, 2, 4, 8, and 16 Hz;

FIG. 10 are graphs comparing pressure differentials and flow rates for an input oscillatory flow which contains two frequencies: 1 Hz and 2 Hz;

FIG. 11 are graphs comparing pressure differentials and flow rates for an input oscillatory flow which contains four frequencies: 1 Hz, 2 Hz, 5 Hz and 9 Hz;

FIG. 12 is a flowchart depicting a method according to another embodiment of the present disclosure;

FIG. 13 is a side elevation view of a device according to another embodiment of the present disclosure;

FIG. 14 is a transparent-view diagram of the device of FIG. 14;

FIG. 15A is a thread-side view of a body of a device according to an embodiment of the present disclosure;

FIG. 15B is a cross-sectional view of the body of FIG. 15A taken along line A-A of FIG. 15A;

FIG. 15C is a perspective view of the body of FIGS. 15A-15B;

FIG. 15D is a side elevation view of the body of FIGS. 15A-15C;

FIG. 16A is a perspective view of a cap of a device according to an embodiment of the present disclosure;

FIG. 16B is a thread-side view of the cap of FIG. 16A;

FIG. 16C is a side elevation view of the cap of FIGS. 16A-16B;

FIG. 16D is a cross-sectional view of the cap of FIGS. 16A-16C taken along line B-B of FIG. 16B;

FIG. 17A is an exploded-view drawing of a device according to another embodiment of the present disclosure;

FIGS. 17B-18E are assembly diagrams of the device of FIG. 17A;

FIG. 18 is a graph depicting forward differential pressure, backward differential pressure, and flowrate signal (corresponding directionally to the respective differential pressures) in a test embodiment; and

FIG. 19 is a graph depicting forward differential pressure, backward differential pressure, and flowrate signal in a test embodiment, wherein the flow was imparted through a diaphragm of the device.

### DETAILED DESCRIPTION OF THE INVENTION

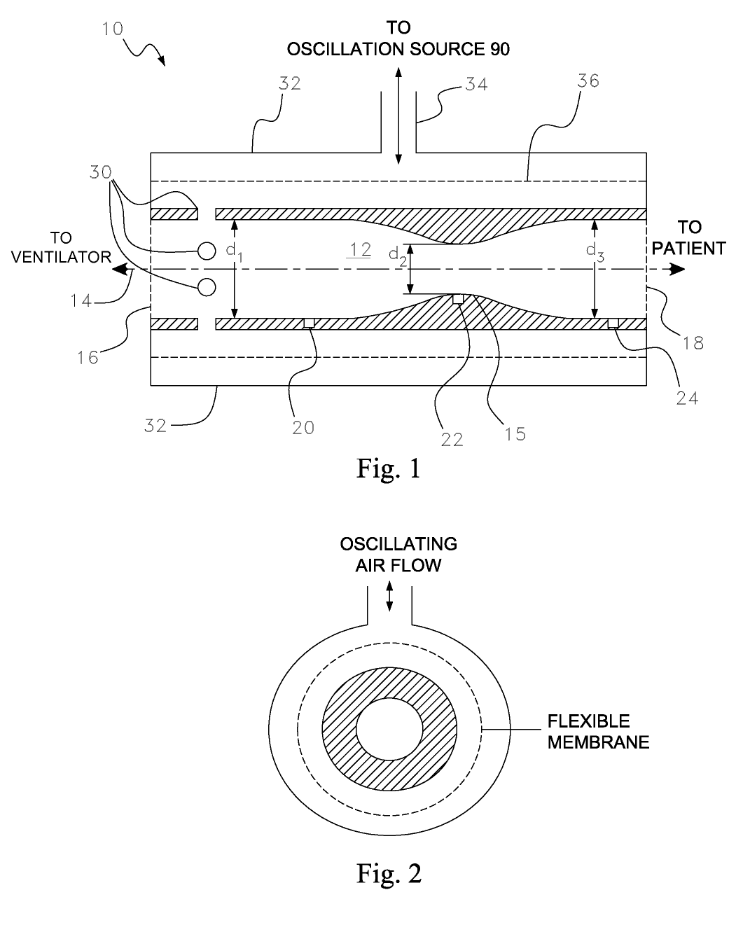

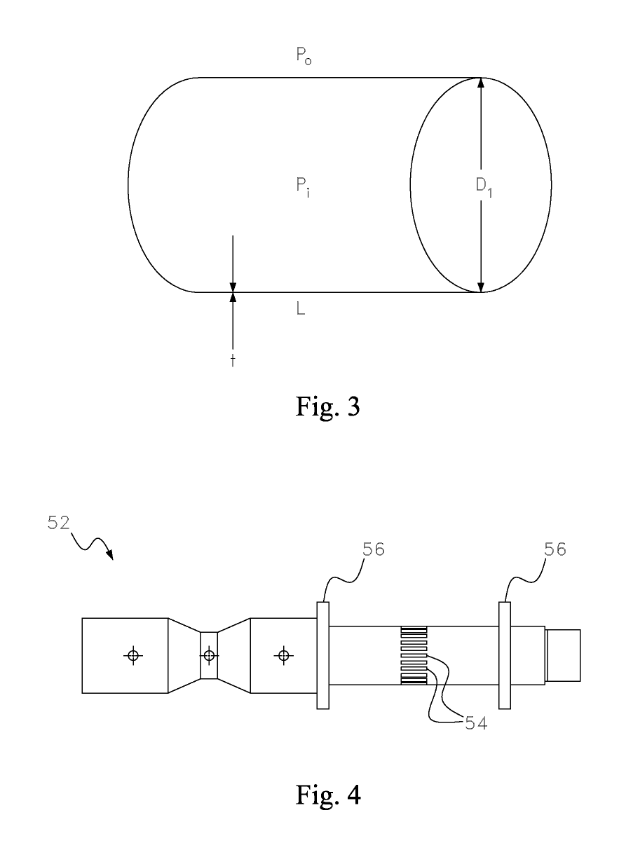

With reference to FIG. 1, the present disclosure may be embodied as a device 10 for measuring the breathing of an individual being mechanically ventilated. The individual may have, for example, an endotracheal tube which is connected to a ventilator, and the ventilator may provide cycles of breathing gas to the patient under pressure to assist or replace the spontaneous breathing of the individual. The ventilator will provide breathing gas at a ventilation frequency. The device 10 is configured for installation between the ventilator and the individual. The device 10 comprises a conduit 12 with a gas flow path 14 for the breathing gas. As such, the device 10 has an inlet 16 and an outlet 18, each of which may be configured for attachment to tubing used for ventilating individuals. For example, the device 10 may be configured for connection to 15 mm connectors which have become a standard for medical breathing tubes.

The conduit 12 is configured as a Venturi tube, having an inlet diameter, d₁, at the inlet 16, an outlet diameter, d₃, at the outlet 18, and a restriction diameter, d₂, at a location (restriction 15) along the gas flow path 14 between the inlet 16 and the outlet 18, each diameter being an inner diameter of the conduit 12. The inlet diameter, d₁, may be the same as the outlet diameter, d₃. Reference is made throughout the remainder of this disclosure to non-limiting embodiments wherein the inlet and outlet diameters are the same. The transition between inner diameters of the conduit 12 may be gradual, as is typical with a venture tube, or more abrupt, such as with a restriction plate with an orifice of diameter d₂, or suitable hybrids of such configurations. The ratio between the diameters d₂:d₁, d₃, the “constriction ratio,” can be any ratio suitable for such measurements. For example, the constriction ratio may be 1:2.

A first pressure transducer 20 is positioned at a location L₁ along the gas flow path 14 where the conduit 12 has the inlet diameter d₁. The location L₁ of the first pressure transducer 20 is positioned on the inlet side of the restriction diameter, d₂, (i.e., between the restriction 15 and the inlet 16). The first pressure transducer 20 is configured such that it can be used to measure the pressure P₁ of the breathing gas within the conduit 12 at location L₁. Suitable pressure transducers are known in the art and may require an orifice at the location L₁ for insertion of a portion (for example, a port) of the transducer. A suitable pressure transducer is a Honeywell® TruStability® SSC Series pressure transducer. Other suitable pressure transducers will be known to those skilled in the art in light of the present disclosure.

A second pressure transducer 22 is positioned at a location L₂ along the gas flow path 14 where the conduit 12 has the restriction diameter d₂. The second pressure transducer 22 is configured such that it can be used to measure the pressure P₂ of the breathing gas within the conduit 12 at location L₂. The second pressure transducer 22 may be the same as or different from the first pressure transducer 20.

A third pressure transducer 24 is positioned at a location L₃ along the gas flow path 14 where the conduit 12 has the outlet diameter d₃. As such, the location L₃ of the third pressure transducer 20 is on the opposite side of the restriction diameter, d₂, portion of the conduit 12 than the first pressure transducer 20. The third pressure transducer 24 is configured such that it can be used to measure the pressure P₃ of the breathing gas within the conduit 12 at location L₃. The third pressure transducer 24 may be the same as or different from one or both of the first and second pressure transducers 20, 22.

The device 10 further comprises a measurement orifice 30 between the inlet 16 and the restriction 15 of the conduit 12. The measurement orifice 30 may comprise multiple orifices 30. The measurement orifice 30 is configured to impart an oscillating pressure on the breathing gas of the gas flow path 14 by way of an oscillatory gas provided by an oscillation source 90. In an exemplary embodiment, the oscillation source provides a gas to the measurement orifice 30 of the device 10 at a pressure which oscillates at an oscillatory frequency Fₒ. The frequency of oscillation Fₒ is selected to be higher than the frequency of the ventilation provided to the individual. In this way, the effect of the oscillatory flow may be isolated from the breathing rate of the individual during analysis. The frequency Fₒ may be, for example, five to ten times (or more) the ventilation frequency. In another example, the frequency Fₒ ranges from approximately 5 Hz to 10 Hz, 15 Hz, 20 Hz, or more. The oscillatory gas may provide an oscillating gas volume of up to 10 mL or more.

In another embodiment, the device 10 further comprises a housing 32 in pneumatic communication with the measurement orifice 30. The housing 32 has a port 34 for receiving oscillatory gas from the oscillation source 90. A diaphragm 36 is disposed in the housing 32 such that the diaphragm 36 isolates the oscillatory gas from the breathing gas. In this manner, the diaphragm 36 communicates the oscillating pressure from the oscillatory gas to the breathing gas without intermixing the gases. The housing 32 may be in any appropriate configuration. FIG. 1 depicts an embodiment wherein the housing 32 is coaxial with the conduit 12. In the depicted embodiment, the diaphragm 36 is also coaxial with the housing 32 and the conduit 12.

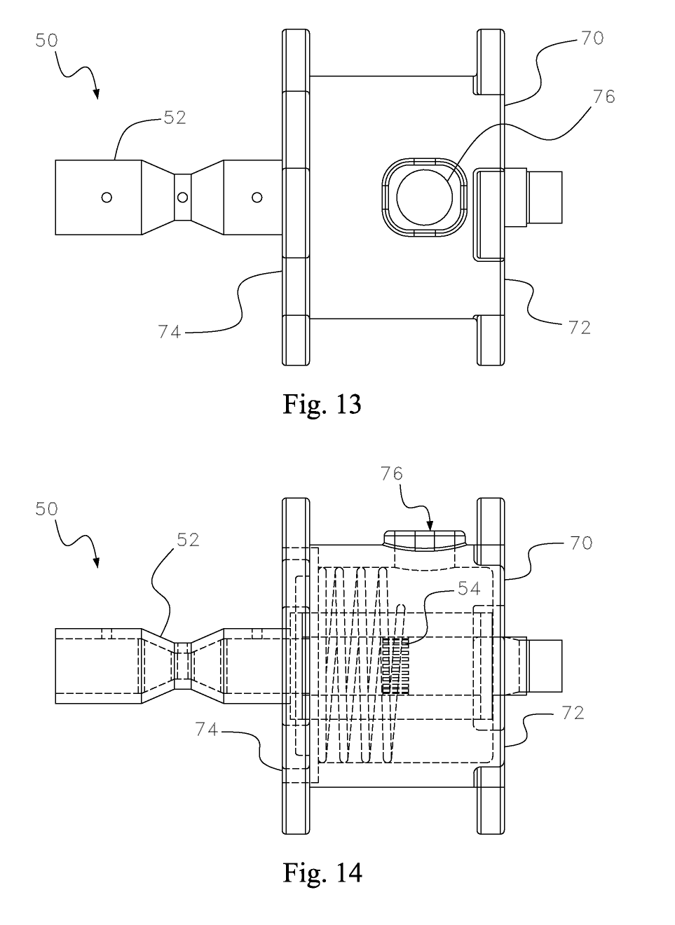

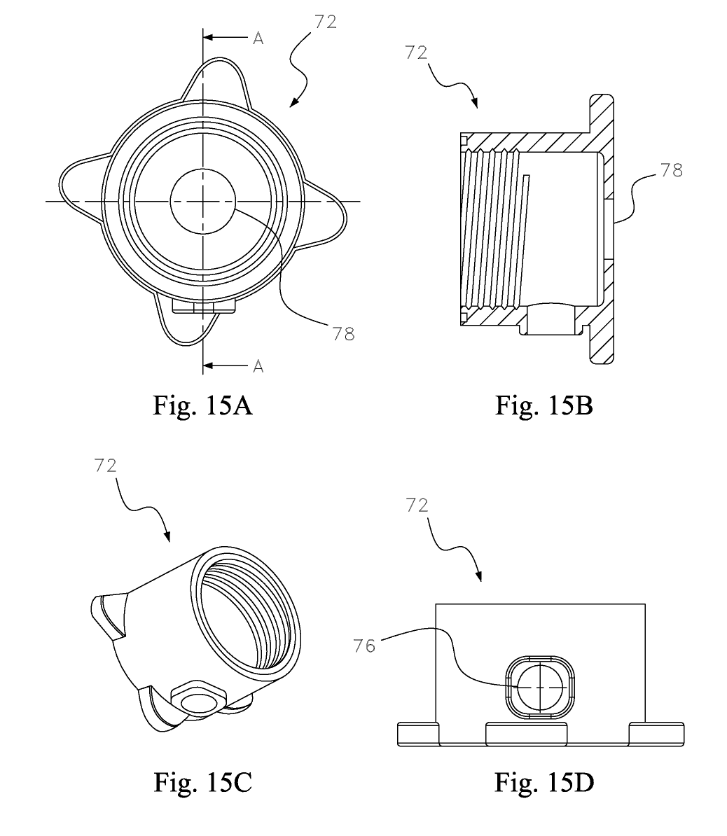

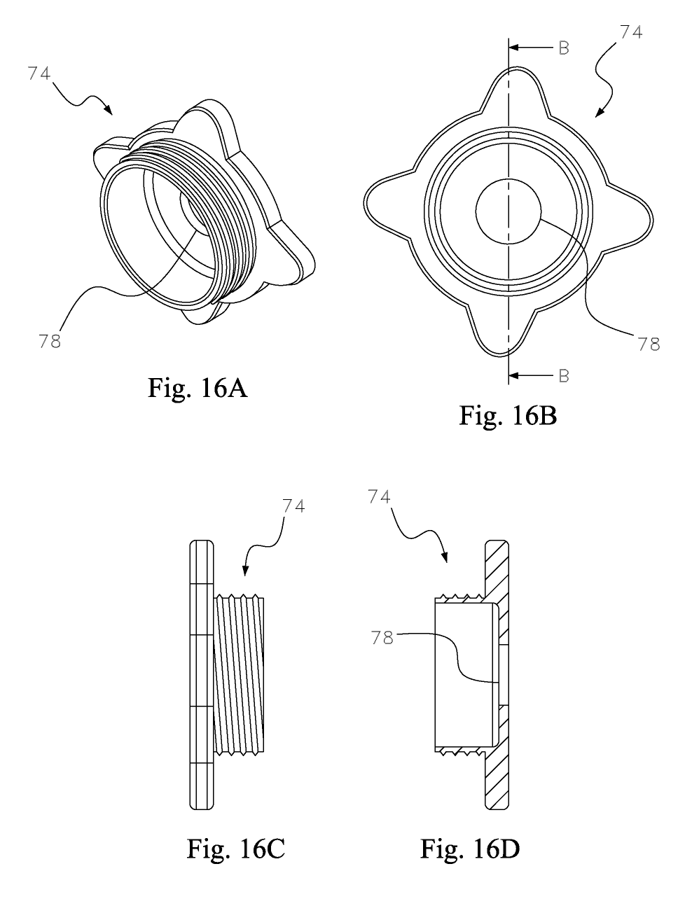

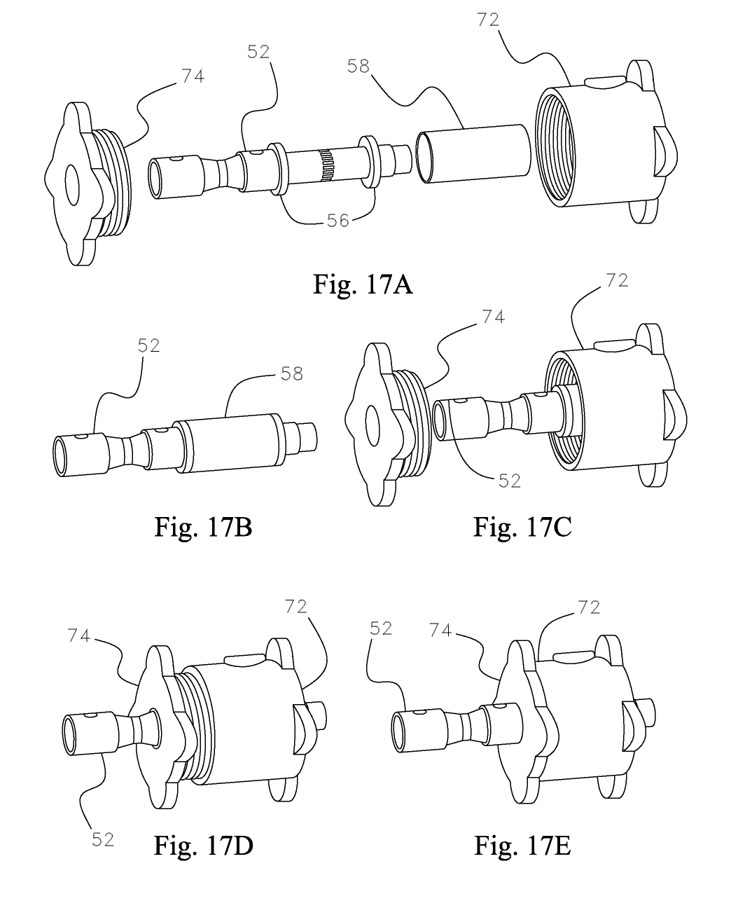

FIGS. 13-14 depict another embodiment of a device 50 wherein the housing 70 is coaxial with the conduit 52 and the diaphragm 58. The housing 70 has a body 72 (see also FIG. 15) and a cap 74 (see also FIG. 16) which are threaded in order to couple with one another. The body 72 has a port 76 for connection to the oscillatory gas source. The body 72 and cap 74 each have a conduit orifice 78 configured for receiving the conduit 52 (see, for example, FIGS. 17A-17E). The conduit 52 has measuring orifices 54 disposed between flanges 56 on which diaphragm 58 is disposed. In this way, movement of the diaphragm 58 caused by oscillatory gas, received through port 76, imparts a corresponding oscillation on breathing gas in the conduit 52 by way of the measuring orifices 54.

The diaphragm 36 may be a flexible barrier, a flexible membrane, a plastic film, a resilient barrier with a bellows, or any other configuration where the oscillating pressure of the oscillatory gas may be transferred through the diaphragm 36 to the breathing gas with little attenuation of the pressure.

The device 10 may further comprise a controller, not shown, in electronic communication with the pressure transducers, 20, 22, 24. The controller is programmed to determine the flow of breathing gas through the conduit 12 using known differential pressure flow measuring techniques. The controller may be further programmed to determine the flow of breathing gas attributable to the individual's breathing (lung function) from the total flow (and further described below).

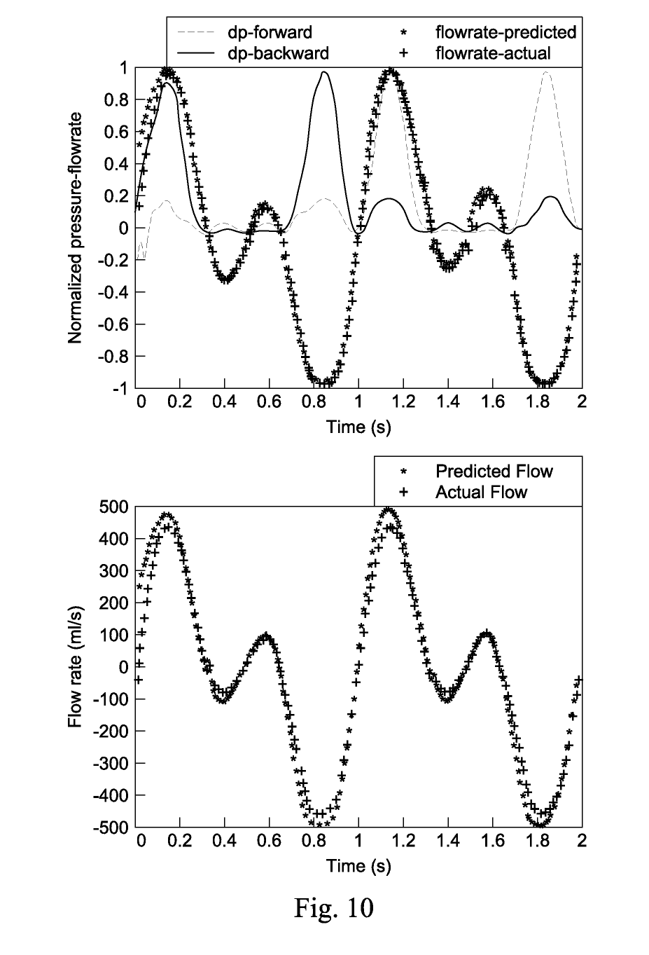

The present disclosure may be embodied as a method 100 for measuring the lung mechanical function of a patient breathing with the assistance of a ventilator (see, e.g., FIG. 10). As previously described, the individual may have, for example, an endotracheal tube which is in fluid (breathing gas) communication with a ventilator having a ventilation frequency. In this way, a gas flow path extends between the endotracheal tube and the ventilator. The method 100 comprises the step of providing 103 a flow meter in the gas flow path. A flow meter, such as device 10 described above, is introduced into the gas flow path. The flow meter may operate on the principle described above (Venturi effect) or may operate on other flow measuring principles.

The method 100 further comprises the step of causing 106 an oscillation in the pressure of the breathing gas of the gas flow path. For example, an oscillator may provide a gas having an oscillating pressure to the flow meter which causes 106 an oscillation in the pressure of the breathing gas. In an embodiment, the gas from the oscillator may be isolated from the breathing gas. The frequency of oscillation Fₒ of the pressure is selected to be higher than the ventilation frequency. In this way, the effect of the oscillatory flow may be isolated from the breathing rate of the individual. The frequency Fₒ may be, for example, five to ten times (or more) the ventilation frequency. In another example, the frequency Fₒ ranges from approximately 5 Hz to 10 Hz or more.

The method 100 includes the step of using 109 the flow meter to measure the flow of breathing gas. The flow meter is used 109 to measure the flow of breathing gas at a point where the flow of breathing gas includes the imposed oscillatory flow. The method 100 comprises determining 112 the portion of the measured flow of breathing gas which is attributable to the patient's breathing. In this way, lung mechanical function may be determined.

Further detail of the presently disclosed devices is described with reference to a Disposable Impedance Adaptor (“DIA”) embodiment. The DIA comprises a conduit with an internal diameter profile designed to allow the measurement of pressure and flow from appropriately placed pressure transducers, while at the same time having forced oscillations in flow produced via an external oscillating pressure source that applies displacements to a flexible barrier separating the flow source from the patient's airways. The entrance and exit ports of the DIA match the standard ends of an endotracheal tube and ventilator tubing, and so it can be snapped into place. A schematic of another embodiment of the DIA is shown in FIG. 6. A flexible barrier physically isolates the patient's lungs from the oscillating pressure source in order to ensure sterile conditions for the patient. The measurement of the pressure (P₁) at the entrance to the endotracheal is provided by a gauge pressure transducer (e.g., a piezoresistive strain gauge, etc.) The flow (V′) entering the endotracheal tube is measured using the Venturi effect. Specifically, measurement of pressure at three sites (P₁, P₂, and P₃ in FIG. 6) will be used in pairs to determine flow from the differences in these pressures.

An external oscillating pressure source cooperates with the DIA to oscillate the flexible barrier in the DIA with displacements of, for example, several tens of milliliters at frequencies up to 20 Hz or more. Electronic components for transducer excitation and signal conditioning for the three pressure transducers, filtering, and analog-to-digital conversion, and software for processing the measured pressure signals to determine parameters of respiratory mechanical function may be provided in certain embodiments.

Measuring the flow (V) and associated pressure (P) oscillations at the entrance to the endotracheal tube allows the calculation of the impedance of the patient's respiratory system, Zᵣₛ, according to:

Zᵣₛ(f)=P(f)V′(f) (1)

Where the argument (f) indicates the Fourier transform. A mathematical model may be fit to the measurements of Zᵣₛ to allow evaluation of parameters of physiological importance (notably the flow resistance of the airways and the stiffness of the respiratory tissue to inflation).

By using oscillations that are sufficiently far above those contained in the spontaneous breathing waveform (i.e. above about 5 Hz), Zᵣₛ can be determined even if the patient is making breathing efforts. This is crucial because some patients frequently do make such efforts even when in the depths of their disease. With measurements of Zᵣₛ over a frequency range of, for example, 5-20 Hz, it is then possible to determine the respiratory elasticity (Eᵣₛ) which can be used as a means of following changes in the degree of lung recruitment.

The flow entering the patient is determined on the basis of the Bernoulli effect as it is manifest in the difference between pressures P₁ and P₂ under the assumption that the actual flow resistance between the two pressure measurement sites is negligible. That is, if r₁ and r₂ are the conduit radii at the two measurement sites then the driving pressures (which are assumed equal) at the two sites are underestimated as a result of the Bernoulli effect by the respective amounts:

ΔPb₁=ρ(V′/πr₁²)²/2g (2)

ΔPb₂=ρ(V′/πr₂²)²/2g (3)

where ρ is the density of air and g is the acceleration due to gravity. The difference between P₁ and P₂ is thus equal to the difference between equations 2 and 3, the only unknown in which is V′. This allows V′ to be determined from P₁ and P₂. The same procedure will provide the flow between sites P₂ and P₃, which can be used as a check on the accuracy of measurement if the displacement of the flexible window is also known; the rate of window volume displacement should equal the sum of the oscillatory flow between P₂ and P₁ and between P₂ and P₃. P₂ itself, once corrected for ΔPb₂, provides the pressure for use in the calculation of Zᵣₛ using equation 1.

In some embodiments, it is advantageous to minimize the patient's “dead space” volume and, for such embodiments, the volume of the device minimized. For example, the device may have a volume (of the conduit) of 100 ml. Similarly, it may be advantageous to design the conduit such that the flow resistance through the DIA is small relative to the airway resistance of a patient (a typical value of the latter being about 2 cmH₂O·s·ml⁻¹). This limits the diameter of the narrow region (i.e., the portion of the conduit having the second inner diameter).

The difference in diameter between the narrow region (second inner diameter) of the DIA and the wide region (first inner diameter) should be sufficient to produce a measurable Venturi effect with which to estimate flow through the device. The section below, under the heading “Operating Principle,” shows how the pressures P₁, P₂, and P₃ can be used to accurately estimate flow.

The DIA can be mass-produced from, for example, molded plastic components and peizoresistive pressure transducers. As such, the device may be both disposable and affordable. The DIA will thus allow the forced oscillation technique to be applied in mechanically ventilated patients in a manner that is convenient, and which reduces the risk of infections being passed from the device to the patient. The device will make it possible to continually monitor respiratory mechanical function easily and safely in ventilated patients regardless of whether they are completely passive or using their own respiratory muscles to breath partially on their own.

Operating Principle

Where the intended use of the present device is in ventilator applications, the device is preferably compact, has small dead space, is easy to install and maintain, and is inexpensive. Among various types of flow meters, the pressure-based Venturi flow meter is advantageous. Traditionally the Venturi flow meter is used for steady flow measurements and commercial products are available for various applications. However, its use for unsteady flow measurements is rare, especially for medical-related applications.

The measurement technique is based on the unsteady Bernoulli equation. For incompressible flow in a pipe in the absence of gravity and frictional energy loss, the following relation (Bernoulli equation) exists:

$$\frac{\partial φ ( x , r , t )}{\partial t} + \frac{1}{2} {u}^{2} ( x , r , t ) + \frac{P ( x , r , t )}{ρ} = const \qquad ( 4 )$$

where φ is a velocity potential, u is velocity, P is pressure, ρ is fluid density, and x and r are axial and radial location in the tube, respectively. If a flat velocity profile (average velocity equals u) in the tube is assumed, then the average velocity follows the above equation (4), and the flow rate will be:

q(t)=S(x)u(x,t) (5)

Applying equation (4) to two axial positions (x₁ and x₂) in the tube results in:

$$A \frac{d q ( t )}{d t} + {Bq}^{2} ( t ) + \frac{δ P ( t )}{ρ} = 0 \qquad ( 6 )$$

where A and B are geometrical factors:

$$\begin{matrix}A = {\int }_{x 1}^{x 2} \frac{d x}{S ( x )} & ( 7 ) \\ B = \frac{1}{2} ( \frac{1}{{S}_{2}^{2}} - \frac{1}{{S}_{1}^{2}} ) & ( 8 )\end{matrix}$$

and δP is pressure differential between x₁ and x₂:

δP=P₂−P₁ (7)

For a Venturi meter, constants A and B are known, and the differential δP is measured. Thus by solving equation (6) it is possible to obtain volume flow rate for steady as well as unsteady flows. For steady flow, time rate of change of flow is zero and the flow rate is easily obtained by equation (6).

Simulation

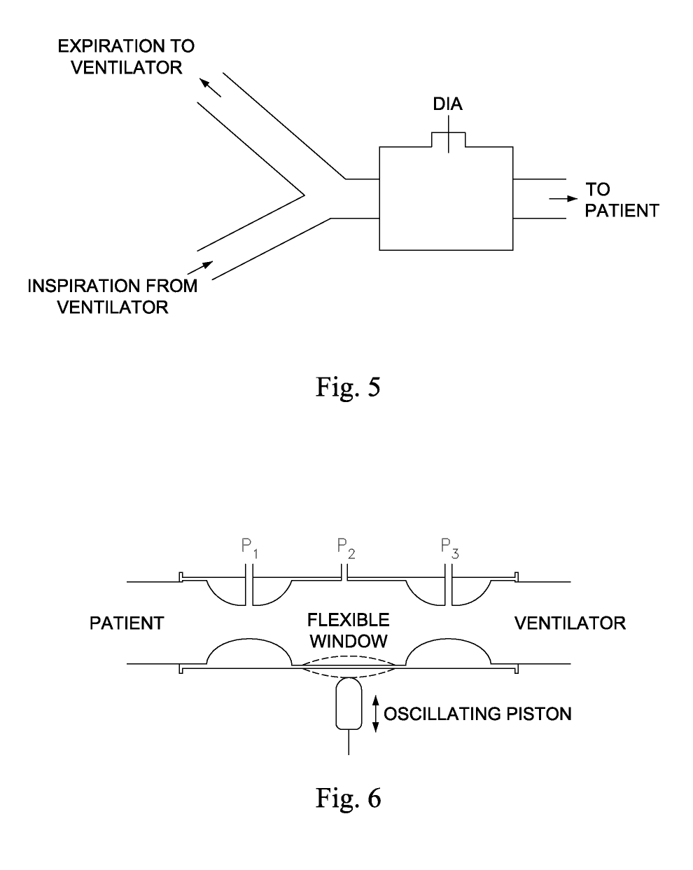

An exemplary symmetric Venturi flow meter was designed using commercial CAD software SolidWorks (ver. 2012, Dassault Systemes). The relative dimensions and a shaded view are shown in FIG. 5. The actual physical dimensions were: normal lumen diameter=18 mm, throat diameter=9 mm, throat length=9 mm, and other dimensions are obtainable using the relative dimensions. The total volume of the model=12.12 cm3. Linear transition through the throat was assumed.

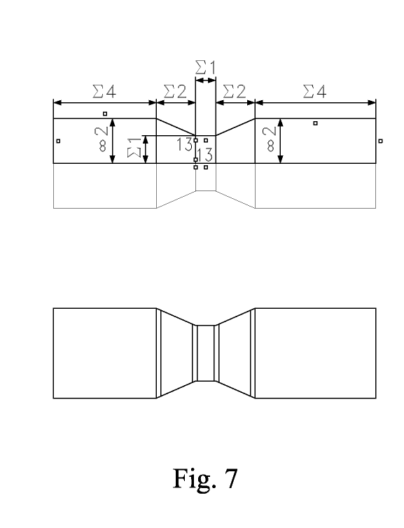

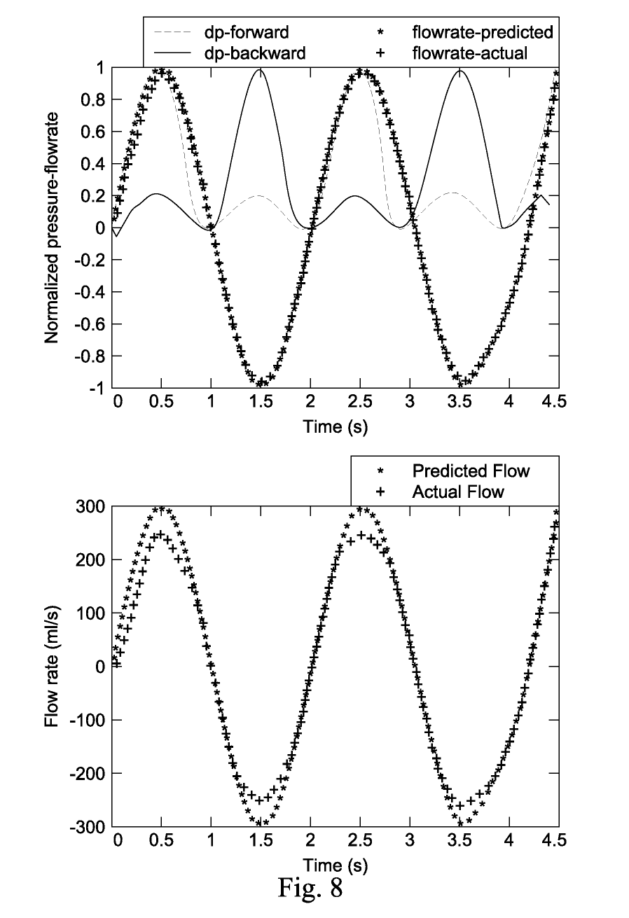

While equation (6) applies at any instant in time in conduit flow, when flow reverses direction, the downstream flow separation can affect the accuracy of the pressure measurement. Therefore, the two pressure measurement ports are advantageously located at the upstream site and throat. For measuring arbitrary unsteady flow including flow reversal, three pressure ports (port 1, 2 and 3, with port 2 in the throat in FIG. 7) were created as shown in FIG. 7. By judicious selection of pressure differentials between the three pressures and solving equation (6), flow rate in both directions can be obtained. An exemplary algorithm for selecting the pressure differential mode (flow direction) follows.

Assume dp₂₁=p₂−p₁, dp₂₃=p₂−p₃, and set a variable f_{sign} to indicate the flow direction: if f_{sign}=1, then positive flow; if f_{sign}=−1, then negative flow.

Exemplary algorithm for selecting pressure differentials and computing flow rate for unsteady flow:
1. Set f_{sign}=1;
2. Assume a small positive initial flow qₒ which flows from port 1 to port 3;
3. At a time step tₙ, compute a small threshold flow value q₁ based on absolute peak flow rates qₚ in previous time steps: qₜ=0.04 qₚ;
4. If |qₙ₋₁|<qₜ and ((qₙ₋₁>0 and |dp₂₁|<|dp₂₃|) or (qₙ₋₁<0 and |dP₂₁|>|dp₂₃|)), set f_{sign}=−f_{sign};
5. If f_{sign}=1, then set δp=dp₂₁;• if f_{sign}=−1, then set δp=dp₂₃;
6. Solve equation (4) to get flow rate at current time step qₙ using 4^{th} order Runge-Kutta method;
7. Repeat steps 3 to 6 to desired time point.

To test the method, single and mixed sinusoidal gas flows through the model were simulated using commercial CFD software Fluent (ver. 14, Ansys Inc.). Fluid was assumed to be a gas with constant density 1.2 kg/m³. Boundary conditions were set as: velocity inlet near the port 1 end and pressure outlet near the port 3 end. A time-varying velocity was given at velocity inlet. The time-varying velocity was either a single sinusoidal function of time, or the sum of several sinusoidal functions with different frequencies. Pressures at ports 1, 2 and 3 were used to compute an ideal flow rate via the above algorithm and compared with the actual values from the CFD solution.

For steady flow with inlet velocity vᵢₙ=1 m/s, the Reynolds number at the throat is Re=2409. A turbulence model was employed in flow simulations. Actual flow rate q_{actual}=254 ml/s, and computed ideal flow rate q_{ideal}=306 ml/s. Discharge coefficient D_{c}=q_{actual}/q_{ideal}=0.83.

For inlet velocity: qᵢₙ(t)=sin(πt), which has a frequency of 0.5 Hz, the pressure differentials and flow rates are shown in FIG. 8. The two pressure differentials alternate between large and small values, corresponding to the reversal of flow direction. The algorithm was able to detect the change in flow direction and assign correct pressure differentials to compute flow rate. The pressure-based predicted “ideal” flow rate closely follows the actual flow rate, with a small amount of over-prediction due to negligence of friction. The mean discharge coefficient is 0.84, which is very close to the value in steady flow. After adjusting for the discharge coefficient, the predicted-adjusted and actual flow rates almost overlap with each other.

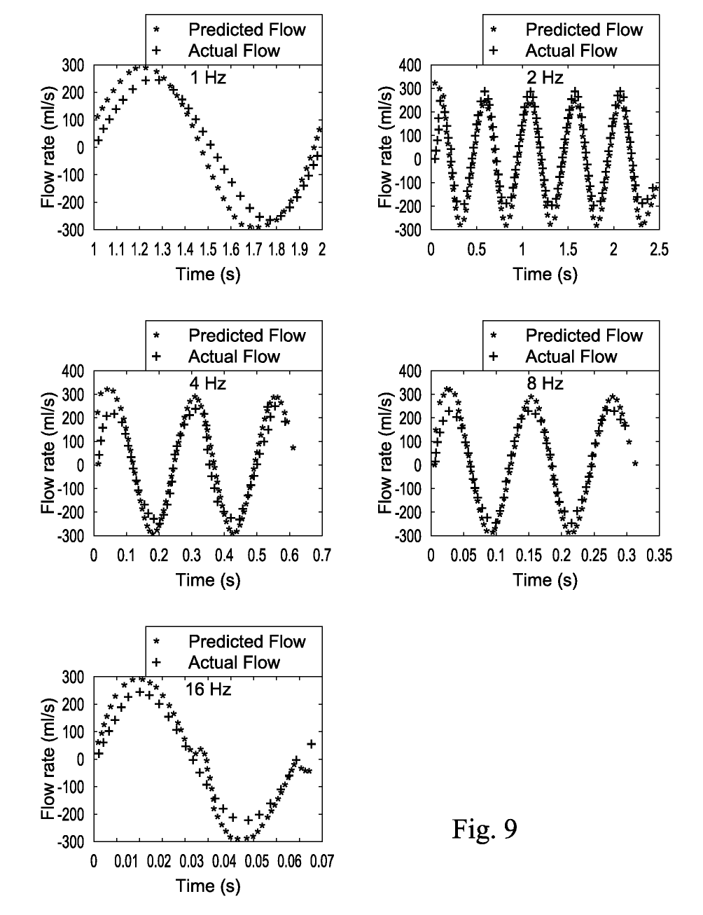

For oscillatory input flow with single frequency of 1, 2, 4, 8 and 16 Hz, the predicted flow rates matched well the actual flow rates (FIG. 9).

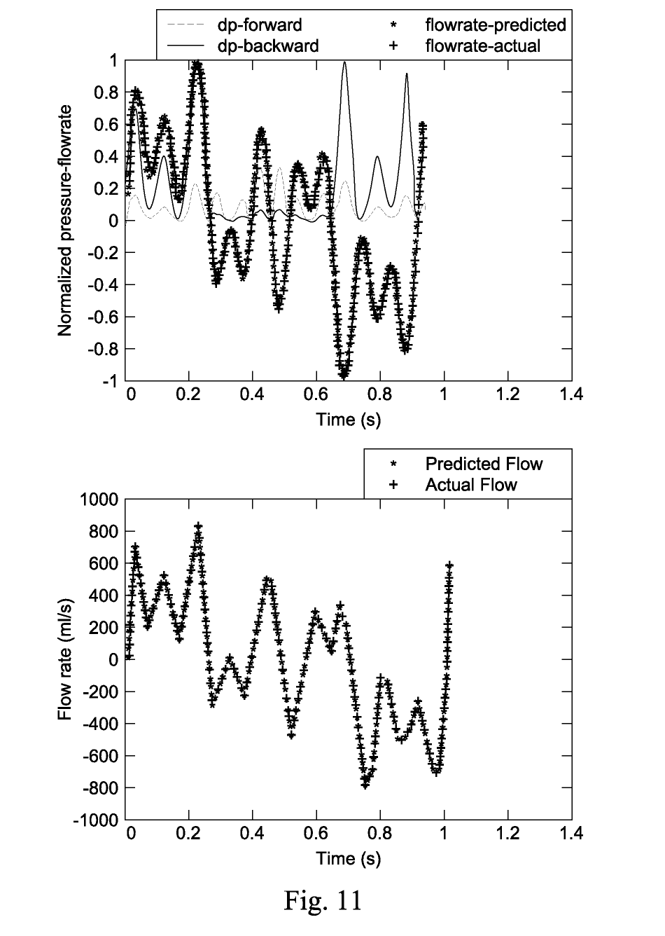

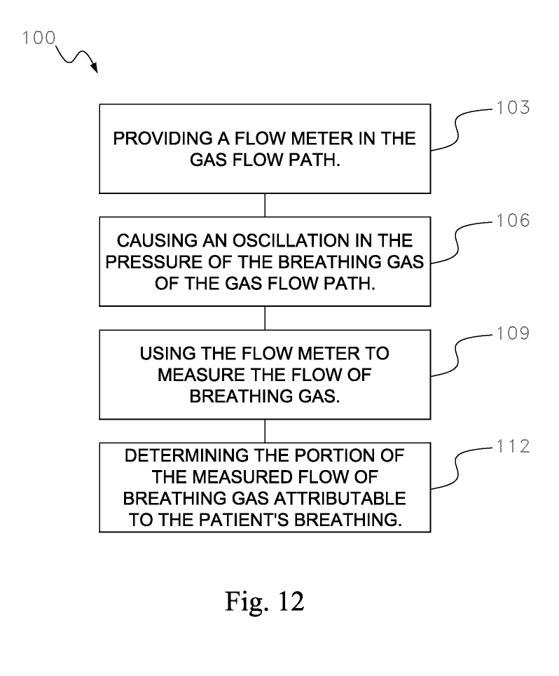

For an input oscillatory flow with two frequency components (FIG. 10): qᵢₙ(t)=sin(2πt)+sin(4πt), and four frequency components (FIG. 11): qᵢₙ(t)=sin(2πt)+sin(4πt)+sin(10πt)+sin(18πt), good matches between predicted and actual flow rates were obtained.

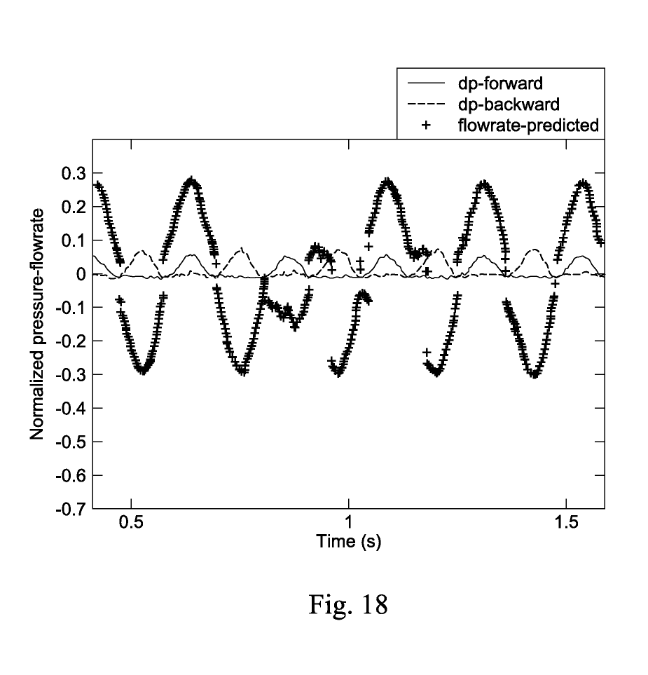

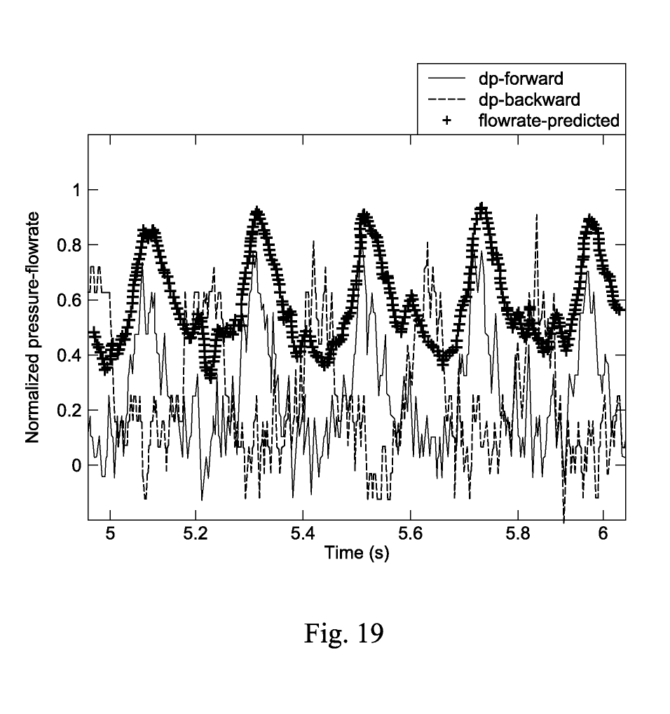

An exemplary embodiment was built for testing purposes. The exemplary device was used to determine the flow rate of a 5 Hz gas flow from the inlet to the outlet. The sampling rate was approximately 625 samples per second. The test apparatus was successfully used to calculate a flowrate from the pressure signals (flowrate, forward differential pressure, and backward differential pressures are shown in FIG. 18). Similarly, the test device was successfully used to show that a flowrate could be calculated on an oscillation imparted through a diaphragm (FIG. 19).

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. A device for measuring the breathing of an individual mechanically ventilated with a breathing gas at a ventilation frequency, comprising:
a conduit having a gas flow path between an inlet and an outlet, the conduit having an inlet diameter at the inlet, an outlet diameter at the outlet, and a restriction diameter at a restriction along the gas flow path between the inlet and the outlet;
a first pressure transducer at a location of the conduit having the inlet diameter and between the restriction and the inlet;
a second pressure transducer at a location of the conduit having the restriction diameter;
a third pressure transducer at a location of the conduit having the outlet diameter and between the restriction and the outlet; and
a measurement orifice between the inlet and the first pressure transducer, the measurement orifice configured to impart an oscillating pressure on the breathing gas of the gas flow path by way of an oscillatory gas provided by an oscillation source.

2. The device of claim 1, wherein the inner diameter is equal to the outlet diameter.

3. The device of claim 1, further comprising:
a housing in pneumatic communication with the measurement orifice and having a port for receiving the oscillatory gas from the oscillation source; and
a diaphragm disposed within the housing and configured to isolate the oscillatory gas from the breathing gas.

4. The device of claim 3, wherein the diaphragm is a flexible barrier.

5. The device of claim 3, wherein the diaphragm comprises a bellows.

6. The device of claim 3, wherein the diaphragm is an elastomeric barrier.

7. The device of claim 1, further comprising a controller programmed to determine the flow of breathing gas in the conduit.

8. The device of claim 7, wherein the controller is further programmed to determine the flow of breathing gas attributable to the individual's spontaneous breathing.

9. A method of measuring lung mechanical function of an individual breathing a breathing gas at a ventilation frequency via a gas flow path extending between an endotracheal tube and a ventilator, the method comprising the steps of:
providing a flow meter in the gas flow path, wherein the flow meter has a conduit with a proximate side and a distal side;
causing an oscillation in the pressure of the breathing gas using a oscillatory gas from an oscillation source with an oscillatory frequency, wherein the oscillatory frequency is higher than the ventilation frequency;
using the flow meter to measure the flow of breathing gas; and
determining the portion of the measured flow of breathing gas attributable to the breathing of the individual to measure the lung mechanical function of the individual.

10. The method of claim 9, wherein the oscillatory frequency from about 5 Hz to about 20 Hz.

11. The method of claim 10, wherein the oscillatory frequency is 5 Hz.

12. The method of claim 11, wherein the oscillatory frequency ranges from five times the ventilation frequency to twenty times the ventilation frequency.
